(19) United States
(12) Patent Application Publication
(10) Pub. No.: US 2006/0280753 A1
(43) Pub. Date: Dec. 14, 2006

(54) Composition and Method For Obtaining A Nutritional Food Product Using Solid Substrate Fermentation

(76) Inventor: Peter McNeary, Brattleboro, VT (US)

Correspondence Address: WILLIAM J. SAPONE;COLEMAN SUDOL SAPONE P.C., 714 COLORADO AVENUE, BRIDGE PORT, CT, 06605, US

(21) Appl. No.: 11/423,623

(22) Filed: Jun. 12, 2006

Related U.S. Application Data
(60) Provisional application No. 60/689,290, filed on Jun. 11, 2005.
(60) Provisional application No. 60/793,037, filed on Apr. 18, 2006.

(51) Int. Cl. A61K 36/09; A61K 36/06

(52) U.S. Cl. 424/195.15

(57) ABSTRACT

An improved food product has an enhanced nutritional profile by utilizing solid substrate fermentation and ultraviolet irradiation. A solid substrate is mixed with water and other nutrients. The mixture is then sterilized and cooled before aseptically adding UV irradiated mushroom mycelium and maintaining the temperature so as to encourage the growth of the irradiated mycelium onto the surface of the substrate. Other materials capable of carrying out fermentation reactions can also be included such as yeast. Once colonization is complete, the mixture is heated to stop the growth process, and to begin the removal of water through evaporation. Evaporation of water continues until a sufficient moisture level is achieved such that the mixture can be further processed by milling into a fine flour type consistency. The flour has increased protein and carbohydrate content as well as an enhanced vitamin and mineral profile as a result of the fermentation process, with high vitamin D content attributable to the UV irradiation process.

### CROSS REFERENCE TO RELATED APPLICATIONS

This application claims priority in U.S. Provisional Patent application Ser. No. 60/793,037 filed Apr. 18, 2006, and Ser. No. 60/689,290 filed Jun. 11, 2005.

### FIELD OF THE INVENTION

This invention relates to a nutritional food product for use in foods and beverage drink mixes, and more particularly to a fermented mycelium based nutritional product.

### BACKGROUND

Mushrooms are usually produced by first preparing a substrate, such as corn, rice, millet or rye, prepared by soaking the grain in water and sterilizing the substrate before inoculation with mushroom spores or mushroom mycelia. Mycelia are the filamentous hyphae of a mushroom that collect water and nutrients to enable mushrooms to grow. The inoculated substrate is then held to promote colonization of the mycelia, at which point the mycelia-laced grains become “spawn”. This is usually done in individual spawn bags. The substrate provides the nutrients necessary for mycelium growth. The mycelium-impregnated substrate then develops under controlled temperature and moisture conditions, until the hyphae of the mycelium have colonized the substrate. The mycelium enriched product usually is harvested after about four to eight weeks from the beginning of the process, with the contents of the spawn bag possibly processed into dry powdered product.

This invention creates an improved food product with an enhanced nutritional profile by utilizing solid substrate fermentation and ultraviolet irradiation. A carrier or carriers is mixed with water and other nutrients. The mixture is then sterilized and cooled before aseptically adding mushroom mycelium and maintaining the temperature so as to encourage the growth of the mycelium onto the surface of the substrate. Other materials capable of carrying out fermentation reactions can also be included such as yeast. Once colonization is complete the mixture is heated again to stop the growth process, and to begin the removal of water through evaporation. Evaporation of water continues until a sufficient moisture level is achieved such that the mixture can be further processed by milling into a food product with a fine flour type consistency. Either the mixture while growing, or the dried product is additionally treated to UV irradiation. The food product thus has increased protein and carbohydrate as well as an enhanced vitamin and mineral profile as a result of the fermentation process, with a high vitamin D content attributable to the UV irradiation process.

### SUMMARY OF THE INVENTION

It is an object of the present invention to provide a food product for use in foods and beverages which is high in nutritional values, particularly vitamin D.

It is another object to provide a nutritional food product which is a high quality protein source offering many or all of the essential amino acids, yet which is heat stable, odorless, tasteless and will not change the overall taste, consistency, mouth feel or flavor profile of the food product that it is fortifying.

These and other objects of the present invention are achieved by a food product comprising a vitamin fortified mycelium material obtained by colonization of the mycelium on a solid substrate.

The product is obtained by a method comprising the steps of preparing a substrate, such as corn, rice, millet or rye, by soaking the grain in water, sterilizing the substrate, obtaining a mushroom mycelium, and inoculating the substrate with the mushroom mycelium. The inoculated substrate is incubated for about 4-6 weeks at about 65-70° F., allowing the mycelium to completely colonize the mixture. After incubation, the mixture is dried to an appropriate level for use in a particular product. During incubation and/or after drying, the product is subjected to UV irradiation. The product may be dried to the point where a dry millable solid is made so as to produce, for example, milled flour, that can be blended into a number of food products.

Utilizing the irradiated mycelium based food product substantially enhances the vitamin content of the obtained material, having a high vitamin D content, on the order of doubling the content of vitamin D over non-irradiated mycelium.

### DETAILED DESCRIPTION OF THE INVENTION

The present invention relates to method for obtaining a nutritionally enhanced food product using solid substrate fermentation, and the product produced thereby. The solid substrate can be grain and/or legume, vegetable, fruit, nut, seaweed or algae based in origin and include additional nutrient sources for fortifying the food product, such as purified cellulose or other nutrients that provide a health benefit. Providing specific nutrients admixed with the solid substrate enables these nutrients to be integrated, to a certain extent, in the food product.

The fermentation relies on the use of a fungal material such as mycelium in the vegetative state which can colonize the solid substrate to product a nutritionally enhanced product. Other materials capable of carrying out fermentation reactions can also be included to enhance the nutritional value such as yeast. The product can then be dried and milled to produce a flour-like product to be utilized as a food ingredient to produce a variety of nutritionally enhanced food, beverage, nutraceutical and animal feed products.

In the present invention, the food product is subjected to UV irradiation during processing, being irradiated with UV light for a time sufficient to enhance the vitamin D content thereof. By utilizing UV irradiation, the food product has a substantially increased level of vitamin D. Preferably, the food product is irradiated with UV radiation, specifically Ultraviolet-B (UV-B), a section of the UV spectrum, with wavelengths between about 280 and 320 nm, or Ultraviolet-C (UV-C), with wavelengths between about 200 and 280 nm. It is believed that the additional Vitamin D is obtained through the conversion of ergosterol due to the UV irradiation. The time will normally be between 5 minutes and 12 hours, more preferable, 2 hours. The time may be the same or increased when the irradiation occurs during the growing process, though the UV irradiation can occur during both periods.

The solid substrate consists of a grain, legume, vegetable, fruit, nut, seaweed, algae, a cellulose source or mixtures thereof, which is mixed with water and other nutrients utilized during the fermentation process. The mixture is then sterilized to create a sterile microbiologically free substrate, normally by treating under pressure, at a suitable sterilizing temperature, for example, about 10-30 psi, and a temperature of from about 220-300° F.

Mushroom mycelium is then aseptically added to the substrate. This is normally done through inoculation, and may proceed within a bag, as discussed previously. The mixture is then maintained at a temperature specific to the optimum growth temperature for the particular mycelium, to provide the optimum growth conditions for the mushroom mycelium, generally around 65-75° F . Oxygen, carbon dioxide and nitrogen levels are maintained at specific levels to encourage the fermentation process, which normally takes from 2-8 weeks.

Once the fermentation process is complete, the mixture may be heated to a temperature sufficient to stop the growth process. Moisture is removed from the mixture by continued heating until the product is dry enough to be processed into a form for addition to a food product. For example, it may be dried to the point where it may be milled into a flour-like material to be included in food or feed type products. The product can be dried to varying moisture levels so that the product may also be utilized in other product application such as for use as a meat analogue, where a more moist product would improve blending, and mouth feel, or for beverage applications which may require an even higher moisture content. The resultant product has a variety of functional properties including increased protein and carbohydrate content, and can be further fortified by adjusting the substrate ingredients to increase the levels a variety of B vitamins, other vitamins, minerals, trace minerals in additional to potassium, chromium, and selenium, a powerful antioxidant, with these ingredients also being present in the inventive product.

The present invention consists of utilizing a grain or legume material such as rye, oat, barley, millet, corn, rice, soybean, black bean, kidney bean, navy bean, and/or a vegetable source, fruit source, seed source, nut source, algae or botanical source and the like in conjunction with a refined carbohydrate source such as powdered cellulose, cellulose fiber, hydrolyzed guar gum, carboxymethylcellulose, microcrystalline cellulose and the like. The grain provides a surface or inoculation point for the mycelium to adhere to. It also provides a valuable source of nutrients for the mushroom mycelium to metabolize such as protein, carbohydrate, fat, vitamins and minerals. The grain also provides valuable nutritional benefits to the finished food or feed product. The refined cellulose provides a more bioavailable carbohydrate source for the mycelium. This saves the mycelium the extra added step of having to enzymatically break down the cellulose from the grain source first before the mycelium can metabolize it. Various nutrients can be added to the mixture to improve the growth rate of the mycelium, or to allow the mycelium to incorporate the nutrients into the biomass to achieve a specific functional health benefit in the finished product. Water is then added to a moisture level sufficient to sustain the growth of various mushroom mycelia. The mixture is then heated to a time and temperature sufficient to achieve complete sterility. The mixture is then cooled before aseptically adding the mushroom mycelium. The temperature, oxygen, carbon dioxide and nitrogen levels are maintained to achieve optimum growth rate of the mycelium. After the mycelium has completely colonized the solid substrate the entire mixture is then heated again to stop the fermentation process. The heating process is continued to evaporate the water and lower the moisture level to a point where the mixture can be ground into fine flour or other desired final stage of varying moisture.

The nutritional food has increased protein and carbohydrate content compared to grain processed into a flour or another food product without first having been subjected to the fermentation process by mushroom mycelium. The nutritional food also possesses enhanced functional properties such as increased dietary fiber levels, both soluble and insoluble. Beta glucan profiles of the food product stimulate the immune system in addition to providing cholesterol lowering properties. A wide variety of B vitamins, other vitamins, minerals, trace minerals are present as well as high levels of potassium and selenium. The resulting nutritional food product can be used as a partial or complete replace of any food or feed product containing flour as part of its formulation as well as other food, beverage, nutraceutical or feed product which does not contain flour. The nutritional food product is heat stable, odorless, tasteless and will not change the overall taste, consistency, mouth feel or flavor profile of the food product that it is fortifying. Also, the nutritional food product includes a mixture of beta-glucans and chitin, which performs physiologically as fiber and does not possess adverse effects on mineral status

In particular, the nutritional food product contains choline in an amount of from 50 mg to 1,000 mg per 100 grams of the nutritional food product, which produces mental cognition enhancement and retention of mental cognition function particularly in the elderly. The nutritional food product also contains ergothioneine, a novel metabolite produced by fungi which possesses strong antioxidant and cellular protectant properties.

Examples of food and beverage products include but are not limited to snack foods, chips, pretzels, breads, dough, bakery items, snack food bars, yogurt drinks, meal replacements, medical foods, meat analogues, dietary supplements and feed compositions for animals. The flour, the nutritional food provides many nutritional and functional properties and should be viewed as a functional food ingredient.

Food Product Manufacturing Process

The following discloses in detail the manufacturing process for producing the inventive food product, which may be described as a “myco powder” or “mycoflour”. Many variations can be made by substituting certain ingredients, or changing processing steps which will result in different functional properties in the end product.

| Ingredient | % by Weight: |
|---|---|
| Water | 50.0% |
| Oat groats, dehulled oats | 45.9% |
| Calcium sulphate | 3.0% |
| Pleurotus ostreotus mycelium culture* | 1.0% |
| Sodium selenite | 0.1% |
| Total | 100.0% |

*Pleurotus ostreotus culture, for example only and not in any way limiting the scope of the present invention.

Procedure:
1. Add all of the above ingredients except the Pleurotus ostreotus culture to a bulk sterilization chamber and mix.
2. Sterilize the mixture at a pressure of about 15-18 psi, and about 250° F. for approximately 1 hour.
3. Cool the mixture to approximately 70° F. and aseptically inoculate with the Pleurotus culture. Continue mixing to ensure uniform distribution of the inoculum.
4. Aseptically fill the mixture into propylene ethylene bags equipped with microporous filters and heat seal.
5 . Incubate mixture 4-6 weeks at 65-70° F. allowing the Pleurotus mycelium to completely colonize the contents of the bag.
6. After incubation, open bags and dry contents using appropriate methods, i.e. convection oven, to a moisture content of about 10-13%.
7. After drying, send the mixture through a mill to produce a free flowing flour-like product.
8. Subject the flour like material to UV treatment to convert ergosterol to Vitamin D. The Finished product can then be used as a partial or total replacement for flour providing a variety of functional health benefits.

Alternative method:
1. Add all of the above ingredients except the Pleurotus ostreo culture to a ribbon blender and mix thoroughly.
2. Add mixture to autoclavable propylene ethylene bags equipped with microporous filters.
3. Sterilize entire bag including contents at about 15-18 psi and at about 250° F. for approximately 1 hour.
4. Cool the mixture to approximately 70° F. and aseptically inoculate with Pleurotus culture.

5. Seal bags and incubate 4-6 weeks at 65-70° F.

6. The remaining processing steps are identical to the ones outlined above.

Nutritional Analysis of Exemplary Dried/Milled MycoFlour

| | | % US RDI (DRV |
|---|---|---|
| | | where indicated) per 100 |
| | per 100 grams | grams |
| Moisture | 2.43 grams | |
| Protein (see amino acid | 15.6 grams | 31.20% DRV |
| profile below) | | |
| Fat | 3.33 grams | 0.05% DRV |
| Carbohydrates | 65.52 grams | 21.84% DRV |
| Total Dietary Fiber | 15.31 grams | 61% DRV |
| Soluble Dietary Fiber | 2.29 grams | |
| Insoluble Dietary Fiber | 13.02 grams | |
| Niacin | 2.16 mg | 10.80% |
| Vitamin D w/UV | 7422.91 iu | 1855.70% |
| irradiation band B | | |
| Vitamin D w/UV | 7400.88 iu | 1850% |
| irraditiation band C | | |
| Vitamin D w/o | 3942.73 iu | 985% |
| irradiation | | |
| Calcium | 3088.63 mg | 308% |
| Iron | 7.97 mg | 44% |
| Phosphorus | 384.34 mg | 38.40% |
| Copper | 1.61 mg | 80.50% |
| Potassium | 468.80 mg | 13.40% |
| Calories | 354.45 | |
| Sodium | 12.95 mg | 0.54% |
| Zinc | 2.76 mg | 18.40% |
| Manganese | 3.11 mg | 155.50% |
| Magnesium | 136.93 mg | 34.20% |
| Ash | 13.12 grams | |
| Choline | 1.56 mg | |
| Thiamine B1 | 0.15 mg | 10% |
| Pyridoxine | B6 0.153 mg | 13% |
| Riboflavin, | B2 0.34 mg | 5% |
| Folic Acid | 0.056 mg | 0.40% |
| Biotin | 0.02000 mg | 15% |
| Vitamin B12 | 0.3000 ug | 20% |
| (Cyanocobalamin) | | |
| Selenium | 17.5 mcg | 25% |
| AMINO ACID | | |
| PROFILE: | | |
| Alanine | 0.70% | |
| | (per 100 grams) | |
| Arginine | 0.91% | |
| Aspartic Acid | 1.22% | |
| Cystine | 0.35% | |
| Glutamic Acid | 2.72% | |
| Glycine | 0.73% | |
| Histidine | 0.28% | |
| Isoleucine | 0.55% | |
| Leucine | 1.01% | |
| Lysine, total | 0.45% | |
| Methionine | 0.19% | |
| Phenylalanine | 0.70% | |
| Proline | 0.79% | |
| Serine | 0.71% | |
| Threonine | 0.56% | |
| Tryptophan | 0.19% | |
| Tyrosine | 0.42% | |
| Valine | 0.74% | |

The myco powder product of the present invention can be fortified to increase the content of various vitamins and minerals by adjusting the substrate feed for the mycelioum. For example, to increase chromium, a chromium source is added, such as chromium aspartate, chromium chloride, chromium citrate, chromium nicotinate, chromium picolinate, GTF chromium and trivalent chromium. To increase potassium, potassium sources are added, such as potassium sulfate, potassium chloride, potassium citrate, etc. For selenium, a selenium source, such as sodium selenite is used, though of course other sources may also be used.

The nutritional food product of present invention, particularly the mycoflour, is a combination of a good source of high quality protein and a good source of dietary fiber. It is also a product that is low fat and low sodium. The following are examples of how the inventive food product can be used. However, these are illustrative only, and should not be considered to limit the applications for the inventive food product, nor the quantities that may be used in the exemplary examples.

EXAMPLE ONE
Nutrition Bar

| | | |
|---|---|---|
| Soy Protein | 10% | wt |
| Calcium Caseinate | 9% | |
| Whey Protein | 10% | |
| Myco powder | 20% | |
| Cocoa | 8% | |
| Vitamin and Mineral | 2% | |
| Premix | | |
| Salt | 1% | |
| High Fructose Corn Syrup | 30% | |
| Canola Oil | 4% | |
| Water | 5% | |
| Flavor | 1% | |
| Total | 100% | |

EXAMPLE TWO
Beverage

| | |
|---|---|
| Water | 90% |
| Myco powder | 5% |
| Fruit Juice Concentrate | 4% |
| High Intensity Sweetener | 0.325% |
| Citric Acid | 0.30% |
| Flavor | 0.20% |
| Color | 0.025% |
| Potassium Citrate | 0.15% |
| Total | 100% |

EXAMPLE THREE
White Bread

| | |
|---|---|
| Wheat Flour | 44% |
| Myco powder | 10% |
| Sugar | 3% |
| Salt | 2% |
| Shortening | 2% |
| Dough Conditioner | 1% |
| Wheat Gluten | 1% |
| Yeast | 2% |
| Water | 35% |
| Total | 100% |

EXAMPLE FOUR
Dietary Supplement

| | |
|---|---|
| Myco powder | 96% |
| Rice Flour | 4% |
| Total | 100% |

This mixture can be formulated for administration as a tablet, capsule, powder, granule or other form, and also mixed with any number of other dietary supplement ingredients, such as specific vitamins, minerals, extracts, amino acids, etc.

EXAMPLE FIVE
Meat Analogue

| | |
|---|---|
| Soy Protein | 16% |
| Myco powder | 40% |
| Salt | 3% |
| High Fructose Corn Syrup | 30% |
| Canola Oil | 4% |
| Water | 5% |
| Flavor | 2% |

While preferred embodiments of the present invention have been shown and described, it will be understood by those skilled in the art that various modifications can be made without varying from the scope of the invention.

I claim:

1. A nutritional food product comprising a UV irradiated, fermented and driedmycelium/solid substrate mixture.

2. The nutritional food product of claim 1 further comprising a high concentration of vitamin D.

3. The nutritional food product of claim 1 further comprising dietary fiber.

4. The nutritional food product of claim 1 wherein the nutritional food product is heat stable, odorless, tasteless and will not change the overall taste, consistency, mouth feel or flavor profile of a food product in which it is integrated.

5. The nutritional food product of claim 1 further comprising chitin, beta 1:3 and beta 1-6 glucans.

6. The nutritional food product of claim 1 further comprising choline in an amount of from 50 mg to 1,000 mg per 100 grams of the nutritional food product.

7. The nutritional food product of claim 1 further comprising calcium at from 50% to 500% of the RDI for calcium per 100 grams.

8. The nutritional food product of claim 1 further comprising phosphorus.

9. The nutritional food product of claim 1 further comprising manganese at from 40% to 300% of the RDI for manganese per 100 grams.

10. The nutritional food product of claim 1 further comprising ergothioneine.

11. The nutritional food product of claim 1 wherein the solid substrate is selected from the group consisting of grains, seeds, legumes, vegetables, fruits, berries, botanicals, nuts, seaweed, algae and combinations thereof.

12. The nutritional food product of claim 1 wherein the mycelial is selected from the group consisting of pleurotus ostreatus, lentinus edodes, grifola frondosa, agaricus bisporus, agaricus blazei, coriolus versicolor, ganoderma lucidum, hericium erinaceious and combinations thereof.

13. A method for producing a nutritional food product comprising the steps of:
providing a solid substrate;
combining the solid substrate with water and mycelium nutrients;
sterilizing the substrate;
obtaining a mycelium culture;
innoculating the substrate with the mycelium culture to form a mixture;
incubating the mixture for a time and at a temperature to enable the mycelium to grow and ferment the mixture;
drying the mixture to a selected level for use as a food product, and,
subjecting the mixture and/or the food product to UV irradiation.

14. The method of claim 13 wherein the solid substrate is selected from the group consisting of grains, seeds, legumes, vegetables, fruits, berries, botanicals, nuts, seaweed, algae and combinations thereof.

15. The method of claim 13 wherein the mycelium is selected from the group consisting of pleurotus ostreatus, lentinus edodes, grifola frondosa, agaricus bisporus, agaricus blazei, coriolus versicolor, ganoderma lucidum, hericium erinaceous and combinations thereof.

16. The method of claim 13 wherein the mixture and/or food product is irradiated with UV radiation for from 5 minutes to 12 hours.

17. The method of claim 13 further comprising adding nutrients to the substrate from the group consisting of minerals, vitamins, phytonutrients and combinations thereof, for increasing a content on the nutrients in the food product.

18. The method of claim 17 wherein the nutrients are selenium, potassium and chromium.
